(19) United States
(12) Patent Application Publication
(10) Pub. No.: US 2011/0224422 A1
(43) Pub. Date: Sep. 15, 2011

(54) PREPARATION OF CAPECITABINE

(75) Inventors: Rajasekhar Kadaboina, Hyderabad (IN); Sekhar Munaswamy Nariyam, Hyderabad (IN); Veerender Murki, Hyderabad (IN); Raghupati Rama Vinjamuri, Hyderabad (IN); Srinivas Benda, Hyderabad (IN); Shravan Kumar Komati, Hyderabad (IN); Nageshwar Gunda, Hyderabad (IN)

(73) Assignees: DR. REDDY'S LABORATORIES LTD., Hyderabad (IN); DR. REDDY'S LABORATORIES, INC., Bridgewater, NJ (US)

(21) Appl. No.: 13/115,172

(22) Filed: May 25, 2011

Related U.S. Application Data
(63) Continuation of application No. PCT/US2009/066332, filed on Dec. 2, 2009.
(60) Provisional application No. 61/152,864, filed on Feb. 16, 2009.

(30) Foreign Application Priority Data
Dec. 2, 2008 (IN) 3000/CHE/2008

(51) Int. Cl. C07H 19/067

(52) U.S. Cl. 536/28.51

(57) ABSTRACT

The present invention relates to substantially pure capecitabine and processes for the preparation thereof.

### INTRODUCTION

Aspects of the present invention relate to capecitabine and processes for the preparation thereof.

The drug compound having the adopted name “capecitabine” has a chemical name 5′-deoxy-5-fluoro-N-[(pentyloxy)carbonyl]cytidine and has structural formula I.

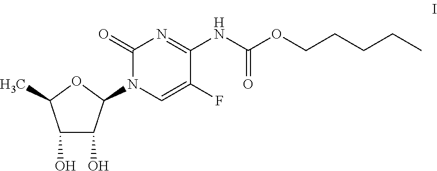

This compound is a fluoropyrimidine carbamate with antineoplastic activity. The commercial product XELODA™ tablets from Roche Pharmaceuticals contains either 150 or 500 mg of capecitabine as the active ingredient.

U.S. Pat. No. 4,966,891 describes capecitabine generically and a process for the preparation thereof. It also describes pharmaceutical compositions, and methods of treating of sarcoma and fibrosarcoma. This patent also discloses the use of ethyl acetate for recrystallization of capecitabine. The overall process is summarized in Scheme I.

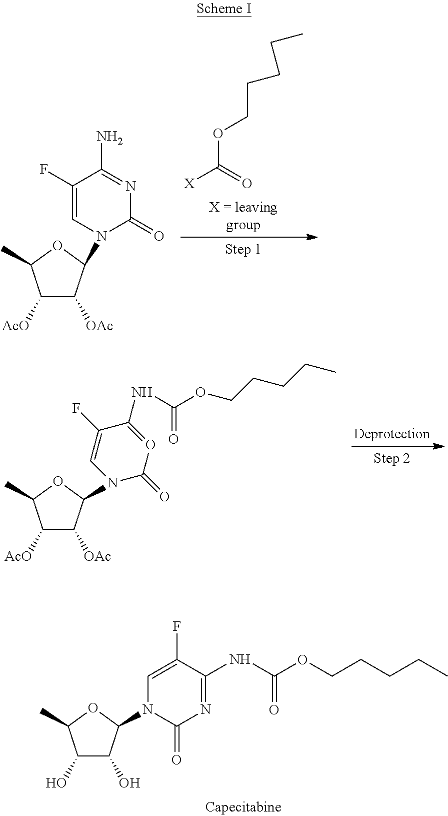

U.S. Pat. No. 5,453,497 discloses a process for producing capecitabine that comprises: coupling of tri-O-acetyl-5-deoxy-β-D-ribofuranose with 5-fluorocytosine to obtain 2′,3′-di-O-acetyl-5′-deoxy-5-fluorocytidine; acylating a 2′,3′-di-O-acetyl-5′-deoxy-5-fluorocytidine with n-pentyl chloroformate to form 5′-deoxy-2′,3′-di-O-alkylcarbonyl-5-fluoro-N-alkyloxycarbonyl cytidine, and deacylating the 2′ and 3′ positions of the carbohydrate moiety to form capecitabine. The overall process is summarized in Scheme II.

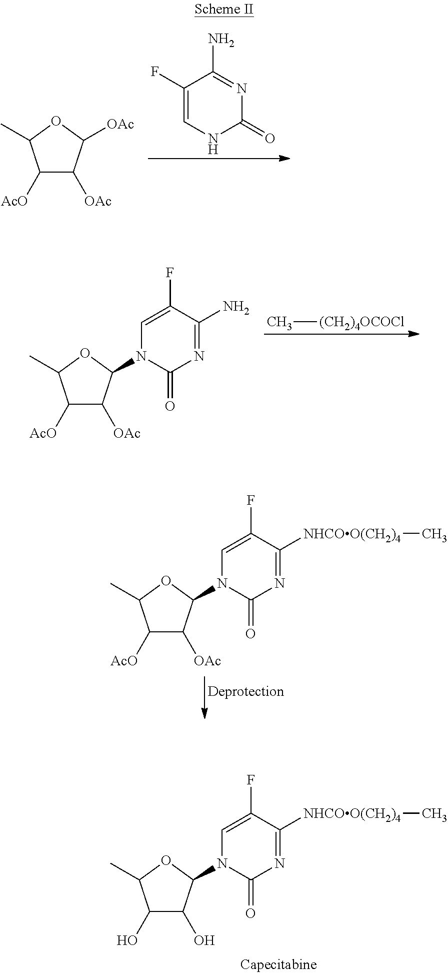

The preparation of capecitabine is also disclosed by N. Shimma et al., “The Design and Synthesis of a New Tumor-Selective Fluoropyrimidine Carbamate, Capecitabine,” Bioorganic & Medicinal Chemistry, Vol. 8, pp. 1697-1706 (2000).

U.S. Pat. No. 7,365,188 discloses a process for the production of capecitabine, comprising reacting 5-fluorocytosine with a first silylating agent in the presence of an acid catalyst under conditions sufficient to produce a first silylated compound; reacting the first silylated compound with 2,3-diprotected-5-deoxy-furanoside to produce a coupled product; reacting the coupled product with a second silylating agent to produce a second silylated product; acylating the second silylated product to produce an acylated product; and selectively removing the silyl moiety and hydroxyl protecting groups to produce capecitabine. The overall process is summarized in Scheme III.

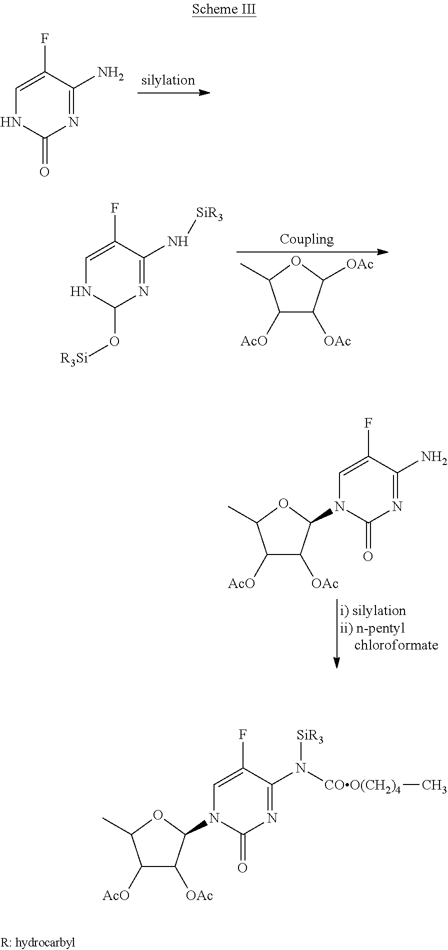

Further, this patent discloses crystallization of capecitabine, using a solvent mixture of ethyl acetate and n-heptane.

International Application Publication No. WO 2005/080351 A1 describes a process for the preparation of capecitabine that involves the refluxing N₄-pentyloxycarbonyl-5-fluorocytosine with trimethylsiloxane, hexamethyl disilazanyl, or sodium iodide with trimethyl chlorosilane in anhydrous acetonitrile, dichloromethane, or toluene, and 5-deoxy-1,2,3-tri-O-acetyl-D-ribofuranose, followed by hydrolysis using ammonia/methanol to give capecitabine. The overall process is summarized in Scheme IV.

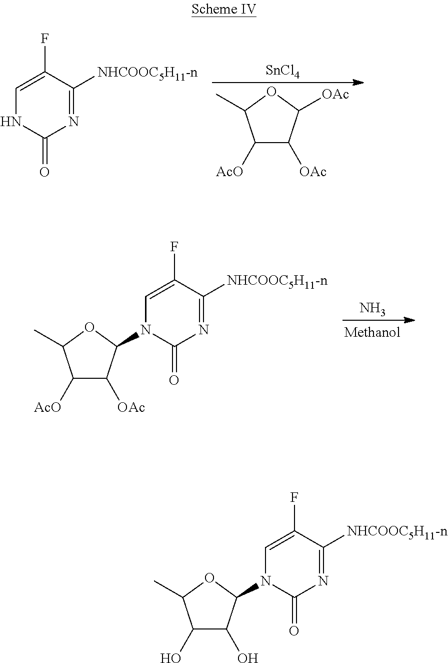

International Application Publication No. WO 2007/009303 A1 discloses a method of synthesis for capecitabine, comprising reacting 5′-deoxy-5-fluorocytidine using double (trichloromethyl) carbonate in an inert organic solvent and organic alkali to introduce a protective lactone ring to the hydroxyl of the saccharide moiety; reacting the obtained compound with chloroformate in organic alkali; followed by selective hydrolysis of the sugar component hydrolytic group using an inorganic base to give capecitabine. The overall process is summarized in Scheme V.

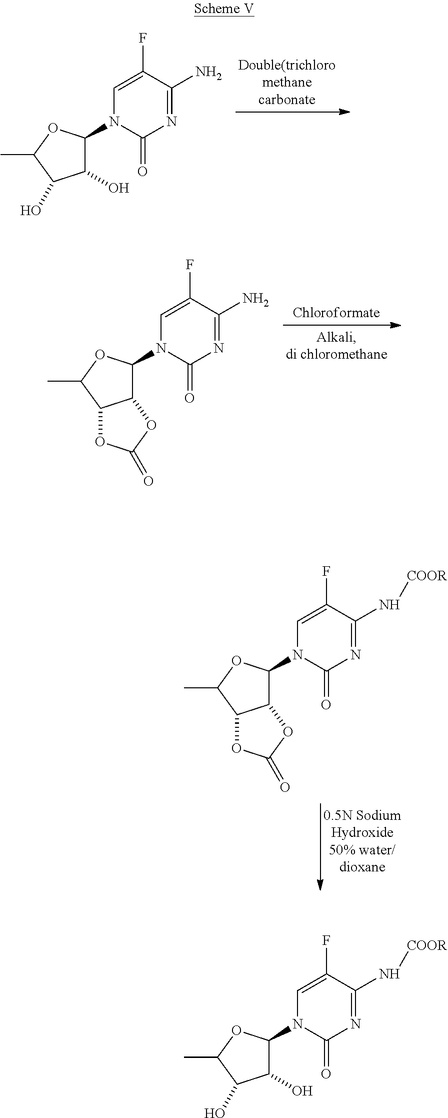

Even though all the above documents collectively disclose various processes for the preparation of capecitabine, removal of process-related impurities in the final product has not been adequately addressed. Impurities in any active pharmaceutical ingredient (API) are undesirable, and, in extreme cases, might even be harmful to a patient. Furthermore, the existence of undesired as well as unknown impurities reduces the bioavailability of the API in pharmaceutical products and often decreases the stability and shelf life of a pharmaceutical dosage form.

### SUMMARY

The present invention includes processes for preparing substantially pure capecitabine comprising:

a) coupling 5-deoxy-D-ribofuranose triacetate of Formula IV,

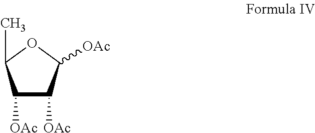

with N,O-(disilylated)-5-fluorocytosine in the presence of a coupling catalyst and an organic solvent to give 2′,3′-di-O-acetyl-5′-deoxy-5-fluorocytidine of Formula III,

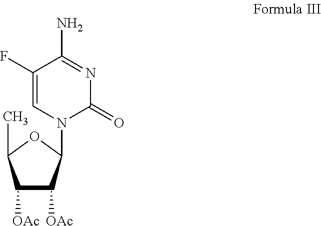

followed by purification of the product;

b) acylating the 2′,3′-di-O-acetyl-5′-deoxy-5-fluoro-cytidine with n-pentyl chloroformate in the presence of a base, such as pyridine, to give 2′,3′-di-O-acetyl-5′-deoxy-5-fluoro-N-[(pentyloxy)carbonyl]-cytidine of Formula II,

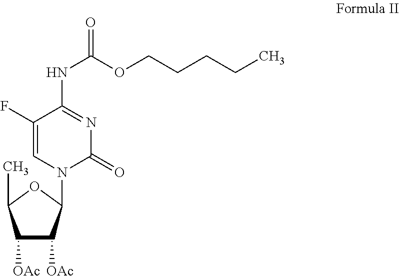

followed by its purification by slurrying in an organic solvent and isolating the purified compound;

c) deprotecting the 2′,3′-di-O-acetyl-5′-deoxy-5-fluoro-N-[(pentyloxy)carbonyl]-cytidine using a base in the presence of an alcohol solvent, to give capecitabine of Formula I; and

d) optionally, further purifying the capecitabine.

In one embodiment, there is provided a process for preparing a pure intermediate of capecitabine, 2′,3′-di-O-acetyl-5′-deoxy-5-fluorocytidine of Formula III,

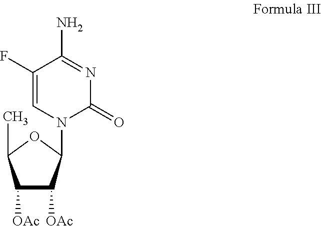

comprising:

i) providing a solution of 2′,3′-di-O-acetyl-5′-deoxy-5-fluorocytidine in a nitrile solvent; and

ii) crystallizing a solid; and

iii) recovering the solid.

In another embodiment, there is provided a process for preparing a pure intermediate of capecitabine, 2′,3′-di-O-acetyl-N-(pentyloxycarbonyl)-5′-deoxy-5-fluorocytidine of Formula II,

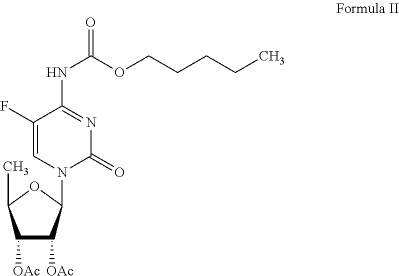

comprising:

i) suspending the 2′,3′-di-O-acetyl-N-(pentyloxycarbonyl)-5′-deoxy-5-fluorocytidine in a suitable liquid, such as, for example, nitriles, ethers or combinations thereof; and

ii) recovering the solid in a pure form.

The present invention includes substantially pure capecitabine having a purity equal to or greater than about 99.8%, and/or having a total content of impurities less than about 0.2%, and/or having each individual impurity being present in an amount less than about 0.1%, as determined using HPLC.

### BRIEF DESCRIPTION OF THE DRAWINGS

FIG. 1 shows an illustrative example of an X-ray powder diffraction pattern for capecitabine, prepared according to Example 6.

FIG. 2 shows an illustrative example of a differential scanning calorimetry curve for capecitabine, prepared according to Example 6.

### DETAILED DESCRIPTION

The term “substantially pure” means a chemical purity equal to or greater than 99.8%, and/or a total content of impurities less than or equal to 0.2%, and/or each individual impurity being present in an amount less than or equal to 0.1%, as determined using high performance liquid chromatography (HPLC). All percentages are expressed herein on a weight basis, unless the context indicates otherwise.

Aspects of the present invention provide processes for preparing substantially pure capecitabine, embodiments comprising at least one of:

a) coupling the compound of 5-deoxy-D-ribofuranose triacetate of Formula IV,

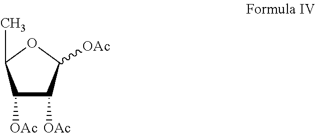

with N,O-(disilylated)-5-fluorocytosine in the presence of a coupling catalyst and an organic solvent, to give 2′,3′-di-O-acetyl-5′-deoxy-5-fluorocytidine of Formula

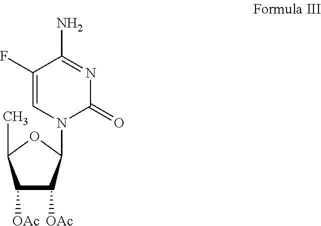

followed by its purification;

b) acylating the 2′,3′-di-O-acetyl-5′-deoxy-5-fluorocytidine with n-pentyl chloroformate in the presence of a base, such as pyridine, to give 2′,3′-di-O-acetyl-5′-deoxy-5-fluoro-N-[(pentyloxy)carbonyl]-cytidine of Formula II,

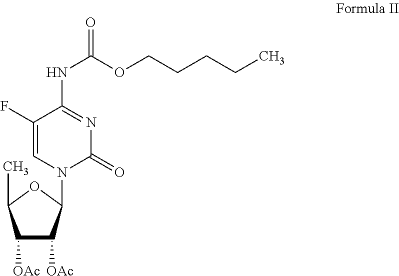

followed by its purification by slurrying in an organic solvent and isolating the purified compound;

c) deprotecting the 2′,3′-di-O-acetyl-5′-deoxy-5-fluoro-N-[(pentyloxy)carbonyl]-cytidine using a base in the presence of an alcoholic solvent to give capecitabine of Formula I; and

d) optionally, purifying the capecitabine.

An overall process according to the present invention is summarized in Scheme 6.

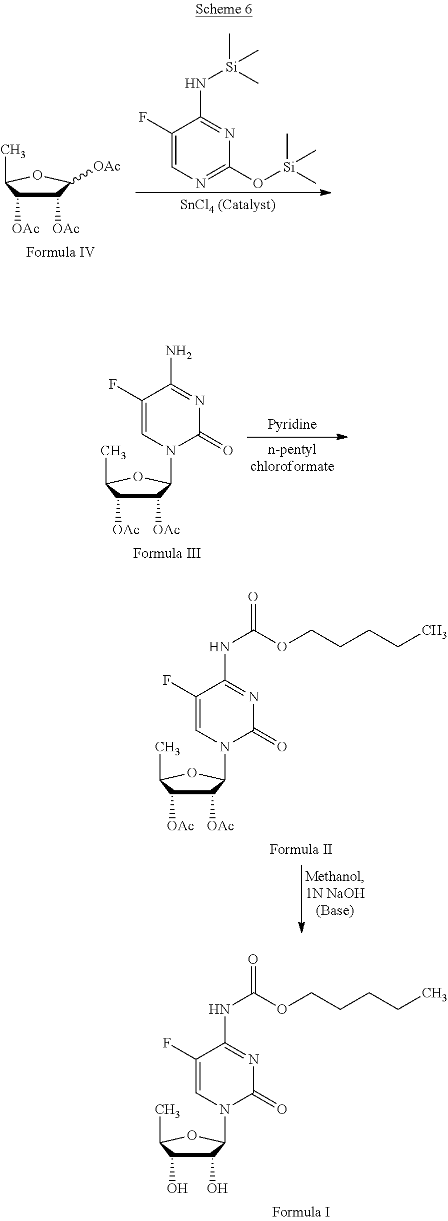

Step a) involves coupling the compound of 5-deoxy-D-ribofuranose triacetate of Formula IV with N,O-(disilylated)-5-fluorocytosine in the presence of a coupling catalyst and a solvent, to give 2′,3′-di-O-acetyl-5′-deoxy-5-fluorocytidine of Formula III, followed by purification of 2′,3′-di-O-acetyl-5′-deoxy-5-fluorocytidine.

Coupling catalysts useful for the coupling reaction include, without limitation, stannous chloride, stannic chloride (SnCl₄) trimethylsilyl trifluoromethanesulfonate, platinum, palladium, or rhodium in concentrated sulfuric acid, and the like.

The amounts of catalyst used in the reaction may be from about 0.5 to about 2 molar equivalents, per molar equivalent of the compound of Formula IV.

The coupling reaction may be carried out in the presence of an organic solvent. Organic solvents useful for the coupling reaction include dichloromethane, 1,2-dichloroethane, chloroform, carbon tetrachloride, toluene, xylene, n-heptane, hexane, and the like.

The coupling reaction may be performed at temperatures about 0-50° C. The coupling reaction may be carried out for any desired time periods to achieve the desired product yield and purity, with time periods from about 1 to 10 hours, or longer, frequently being adequate.

After completion of the coupling reaction, solid 2′,3′-di-O-acetyl-5′-deoxy-5-fluoro-cytidine may be isolated by crystallization from a suitable solvent. Suitable solvents for the isolation of solid 2′,3′-di-O-acetyl-5′-deoxy-5-fluoro-cytidine include, but are not limited to, alcohols, such as, for example, methanol, ethanol, isopropyl alcohol, and the like, and any mixtures thereof.

2′,3′-di-O-acetyl-5′-deoxy-5-fluorocytidine may optionally be purified using any suitable purification technique, such as, for example, recrystallization, filtration, adsorption, and/or extraction. Recrystallization may be achieved by using any suitable method, such as, for example, cooling with or without simultaneous stirring, partial removal of the solvent, seeding, and/or adding an anti-solvent.

In a particular embodiment, purification of solid 2′,3′-di-O-acetyl-5′-deoxy-5-fluorocytidine (Formula III), includes:

i) providing a solution of 2′,3′-di-O-acetyl-5′-deoxy-5-fluorocytidine in a nitrile solvent;

ii) crystallizing a solid from the solution of i); and

iii) recovering purified 2′,3′-di-O-acetyl-5′-deoxy-5-fluorocytidine from ii).

Step i) involves providing a solution of 2′,3′-di-O-acetyl-5′-deoxy-5-fluorocytidine in a nitrile solvent.

Providing a solution can include dissolving solid 2′,3′-di-O-acetyl-5′-deoxy-5-fluorocytidine in a nitrile solvent, or obtaining a solution comprising 2′,3′-di-O-acetyl-5′-deoxy-5-fluorocytidine from a previous synthesis step. Nitrile solvents include, but are not limited to: acetonitrile, propionitrile, and the like

The solution may be prepared at temperatures ranging from about 25° C. to about 100° C., or about 45° C. to about 55° C.

Step ii) involves crystallizing a solid from the solution of i).

Crystallization of the solid from the solution may be obtained by the cooling a solution to temperatures lower than the dissolution temperatures, such as, for example, below about 10° C. to about 35° C., for a period of time as required for a more complete isolation of the product. The exact cooling temperatures and times required for complete crystallization may be readily determined by a person skilled in the art and will also depend on parameters such as concentration and temperature of the solution or slurry.

The crystallization may be enhanced using methods such as cooling, partial removal of the solvent from the mixture, slurring, seeding, adding an anti-solvent to the reaction solution, or any combinations thereof.

Step iii) involves recovering a purified solid from ii).

The solid can be recovered using any techniques such as filtration by gravity or by suction, centrifugation, decantation, and the like.

Wet, recovered, crystallized solid may optionally be further dried, for example, in a tray dryer, vacuum oven, air oven, fluidized bed dryer, spin flash dryer, flash dryer, and the like. The drying may be carried out at temperatures about 35° C. to about 100° C. The drying may be carried out for any desired time periods from about 1 to 20 hours, or longer, until a desired purity has been obtained.

High performance liquid chromatography analysis of a compound having Formula III and associated impurities can be performed using an instrument with an INERTSIL ODS-2 250×4.6 mm, 5 μm column, and the following parameters:

Wavelength of detection: 240 nm by UV.

Flow rate: 1.0 mL/minute.

Buffer: 1.0 mL of glacial acetic acid in 1000 mL of milli-Q water.

Eluent A: 60% buffer, 35% methanol, and 5% acetonitrile (v:v:v).

Eluent B: 15% buffer, 80% methanol, and 5% acetonitrile (v:v:v).

Injection volume: 10 μL.

Column temperature: 40±2° C.

Gradient program:

| | Eluent A | Eluent B |
|---|---|---|
| Minutes | (% v/v) | (% v/v) |
| 0 | 100 | 0 |
| 5 | 100 | 0 |
| 20 | 49 | 51 |
| 30 | 49 | 51 |
| 40 | 20 | 80 |
| 50 | 20 | 80 |
| 55 | 100 | 0 |
| 60 | 100 | 0 |

The purity of 2′,3′-di-O-acetyl-5′-deoxy-5-fluorocytidine (Formula III) that is obtained by the processes of the present invention may be equal to or greater than about 99.5%, and the level of the impurity at about 1.56 RRT (compound of Formula III=1) may be less than about 0.5%, as determined using the above HPLC method.

Step b) involves acylating 2′,3′-di-O-acetyl-5′-deoxy-5-fluorocytidine (Formula III) with an acylating agent in the presence of a base, to give 2′,3′-di-O-acetyl-5′-deoxy-5-fluoro-N-[(pentyloxy)carbonyl]-cytidine (Formula II), followed by its purification.

The quantities of acylating agent, such as, for example, n-pentyl chloroformate, used for the formation of Formula II may range from about 1 to about 4 molar equivalents, per molar equivalent of the compound of 2′,3′-di-O-acetyl-5′-deoxy-5-fluorocytidine.

Suitable bases include, but are not limited to: organic bases such as triethylamine, tributylamine, pyridine, picoline, N,N-dimethylaminopyridine, lutidine, N,N-dimethylaniline, and the like; and inorganic bases such as hydroxides, carbonates, and alkoxides of alkali metals and alkaline earth metals, e.g., sodium hydroxide, sodium carbonate, sodium bicarbonate, sodium methoxide and similar lithium, potassium, calcium, magnesium, and barium compounds.

Step b) may be carried out in the presence of an organic solvent. Organic solvents that may used in include, but are not limited to: halogenated hydrocarbons, such as, for example, dichloromethane, 1,2-dichloroethane, chloroform, carbon tetrachloride, and the like; hydrocarbons, such as, for example, toluene, xylene, heptane, hexane, petroleum ether, and the like; ethers, such as, for example, diethyl ether, dimethyl ether, diisopropyl ether, methyl tert-butyl ether, tetrahydrofuran, 1,4-dioxane, and the like; and any mixtures thereof.

The reaction of b) may be carried out at temperatures about −30° C. to about 45° C., or from about −15° C. to about 30° C.

The reaction may be carried out for any desired time periods to achieve the desired product yield and purity, with times from about 1 to 10 hours, or longer, frequently being adequate.

The reaction mixture comprising 2′,3′-di-O-acetyl-5′-deoxy-5-fluoro-N-[(pentyloxy)carbonyl]-cytidine may be used directly in the next processing step, or a solid product may be isolated from the mixture by repeated distillation processes in the presence of an ether, such as, for example, diethyl ether, dimethyl ether, di-isopropyl ether, methyl tert-butyl ether, tetrahydrofuran, and 1,4-dioxane.

2′,3′-di-O-acetyl-5′-deoxy-5-fluoro-N-[(pentyloxy)carbonyl]-cytidine may be optionally purified using any suitable purification technique, such as, for example, recrystallization, slurrying, filtration, adsorption, and/or extraction.

In a particular embodiment, there is provided a process for a preparing purified intermediate of capecitabine, 2′,3′-di-O-acetyl-5′-deoxy-5-fluoro-N-[(pentyloxy)carbonyl]-cytidine of Formula II, comprising:

i) suspending 2′,3′-di-O-acetyl-5′-deoxy-5-fluoro-N-[(pentyloxy)carbonyl]-cytidine in a liquid; and

ii) recovering a purified solid from i).

Step i) involves suspending the 2′,3′-di-O-acetyl-5′-deoxy-5-fluoro-N-[(pentyloxy)carbonyl]-cytidine in a liquid.

Pure 2′,3′-di-O-acetyl-5′-deoxy-5-fluoro-N-[(pentyloxy)carbonyl]-cytidine may be achieved by slurrying in a liquid, in which the compound is not significantly soluble, followed if desired by cooling the suspension with stirring for a period of suitable time. Suitable organic liquids that may be used for providing a slurry include, but are not limited to: nitriles, such as, for example, acetonitrile, propionitrile, and the like; ethers, such as, for example, diethyl ether, dimethyl ether, diisopropyl ether, methyl tert-butyl ether, tetrahydrofuran, and 1,4-dioxane; and any combinations thereof. As an example, the liquid may be a mixture of acetonitrile and diisopropyl ether.

The slurrying may be carried out at temperatures ranging from about 20-40° C. for suitable times that may range from about 30 minutes to 3 hours, or longer. The exact temperatures and times required for the slurrying may be easily determined by a person skilled in the art and will depend on parameters, such as the concentrations and initial temperatures of the mixtures.

Step ii) involves recovering a solid from i) in purified form.

The solid obtained may be recovered, for example, by filtration by gravity or suction, centrifugation, decantation, and the like. Wet, recovered, crystallized solid may optionally be further dried, for example, in a tray dryer, vacuum oven, air oven, fluidized bed dryer, spin flash dryer, flash dryer, and the like. The drying may be carried out at temperatures about 35° C. to about 100° C. The drying may be carried out for any desired time periods, such as from about 1 to 20 hours, or longer, until a desired purity has been obtained.

High performance liquid chromatography analysis of the compound having Formula II and associated impurities can be performed using an instrument with an INERTSIL ODS-2 250×4.6 mm, 5 μm column, and the following parameters:

Wavelength of detection: 250 nm by UV.

Flow rate: 1.0 mL/minute.

Buffer: 1.0 mL of glacial acetic acid in 1000 mL of milli-Q water.

Eluent A: 60% buffer, 35% methanol, and 5% acetonitrile (v:v:v).

Eluent B: 15% buffer, 80% methanol, and 5% acetonitrile (v:v:v).

Injection volume: 10 μL.

Column temperature: 40±2° C.

Gradient program:

| | Eluent A | Eluent B |
|---|---|---|
| Minutes | (% v/v) | (% v/v) |
| 0 | 100 | 0 |
| 5 | 100 | 0 |
| 20 | 49 | 51 |
| 30 | 49 | 51 |
| 40 | 20 | 80 |
| 50 | 20 | 80 |
| 55 | 100 | 0 |
| 60 | 100 | 0 |

The purity of 2′,3′-di-O-acetyl-5′-deoxy-5-fluoro-N-[(pentyloxy)carbonyl]-cytidine (Formula II) that is obtained by the processes of the present invention may be equal to or greater than about 99.8%, and the level of the impurity at 1.14 RRT (compound of Formula II=1) may be equal to or less than about 0.15% as determined using the above HPLC method. Repeating the above purification process may further enhance the purity.

Step c) involves deprotecting the hydroxyl groups of 2′,3′-di-O-acetyl-5′-deoxy-5-fluoro-N-[(pentyloxy)carbonyl]-cytidine using a base in the presence of an alcohol solvent, to give capecitabine.

Suitable bases for step c) include, but are not limited to hydroxides, carbonates, and alkoxides of alkali metals or alkaline earth metals, e.g., sodium hydroxide, sodium carbonate, sodium bicarbonate, sodium methoxide, and similar lithium, potassium, calcium, magnesium, and barium salts.

The deprotection reaction may be carried out in the presence of an alcohol solvent such as, for example, methanol, ethanol, isopropanol, n-butanol, and the like, and any combinations thereof.

The reaction may be carried out at or below temperatures of about 40° C. The reaction may be carried out for any desired time periods in order to complete the reaction, however, times from about 1 to 10 hours, or longer, are frequently adequate. The amount of base, such as, for example, sodium hydroxide, used for the deprotection reaction may range from about 0.5 to about 2 or more molar equivalents, per molar equivalent of 2′,3-di-O-acetyl-5′-deoxy-5-fluoro-N-[(pentyloxy)carbonyl]-cytidine.

After completion of the reaction, the pH of the reaction mixture may be adjusted to about 3.5-5 by adding an acid, e.g., hydrochloric acid, then the product may be extracted into an organic solvent, which may be concentrated (e.g., distilled under vacuum) to about 2 mL per gram of 2′,3′-di-O-acetyl-g-deoxy-5-fluoro-N-[(pentyloxy)carbonyl]-cytidine. Organic solvents useful for extracting the product include, but are not limited to, halogenated hydrocarbons, such as, for example, dichloromethane and chloroform. The solid capecitabine may be precipitated by combining with an anti-solvent, such as but not limited to hydrocarbons, such as, for example, toluene, xylene, heptane, hexane, and any mixtures thereof.

The capecitabine, obtained according to a process of the present invention as described above, may contain the impurity at about 1.23 RRT at less than or equal to about 0.1%, as determined by HPLC.

Step d) involves optionally further purifying capecitabine using any suitable purification technique, such as, for example, recrystallization, filtration, adsorption, and/or extraction. Recrystallization may be achieved using any suitable method, such as, for example, cooling with or without simultaneous stirring, partial removal of the solvent, seeding, and/or combining with an anti-solvent. For example, capecitabine may be purified by recrystallization from a solution by the addition of an anti-solvent. For the solution, suitable solvents include, but are not limited to: halogenated hydrocarbons, such as, for example, dichloromethane, 1,2-dichloroethane, chloroform, carbon tetrachloride, and the like; alone or in combination with a hydrocarbon, such as, toluene, heptane, hexane, and petroleum ether. The solution may be prepared at temperatures ranging from about 25° C. to 60° C. Suitable anti-solvents include, but are not limited to, hydrocarbons, such as, for example, toluene, xylene, heptane, hexane, petroleum ether, and the like. Upon combination with the anti-solvent, the mixture may be maintained at a temperature lower than the dissolution temperature of the solid, such as, for example, below about 10° C. to about 35° C., for a period of time. The exact cooling temperatures and times required for crystallization may be readily determined by a person skilled in the art and will depend on parameters such as concentrations and temperatures of the solution. The solid may be recovered using any techniques, such as, for example, filtration by gravity or suction, centrifugation, decantation, and the like. Wet, recovered, crystallized solid may optionally be further dried, for example, in a tray dryer, vacuum oven, air oven, fluidized bed dryer, spin flash dryer, flash dryer, and the like. The drying may be carried out at temperatures about 35° C. to about 100° C. The drying may be carried out for any desired time periods, such as from about 1 to 20 hours, or longer, until a desired purity has been obtained.

High performance liquid chromatography analysis of capecitabine and impurities can be performed using an instrument with an INERTSIL ODS-2 250×4.6 mm, 5 μm column, and the following parameters:

Wavelength of detection: 250 nm.

Flow rate: 1.0 mL/minute.

Buffer: 1.0 mL of glacial acetic acid in 1000 mL of milli-Q water.

Eluent A: 60% buffer, 35% methanol, and 5% acetonitrile (v:v:v).

Eluent B: 15% buffer, 80% methanol, and 5% acetonitrile (v:v:v).

Injection volume: 10 μL.

Column temperature: 40±2° C.

Gradient program:

| | Eluent A | Eluent B |
|---|---|---|
| Minutes | (% v/v) | (% v/v) |
| 0 | 100 | 0 |
| 5 | 100 | 0 |
| 20 | 49 | 51 |
| 30 | 49 | 51 |
| 31 | 100 | 0 |
| 40 | 100 | 0 |

Samples are dissolved in a 60:35:5 (v:v:v) mixture of water, methanol, and acetonitrile, to give a capecitabine concentration about 0.6 mg/mL.

The purity of capecitabine that is obtained by the processes of the present invention may be equal to or greater than about 99.8%, and the level of the unidentified impurity at 1.23 RRT may be less than about 0.1%, as determined by HPLC.

In a particular embodiment, there is provided substantially pure capecitabine, wherein the amount of the impurity 5-fluoro-N-[(pentyloxy)carbonyl]-cytidine (at 0.75 RRT, also called Impurity 8), is less than about 0.1%, as determined using HPLC.

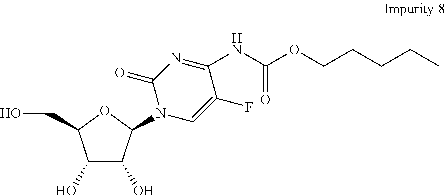

In embodiments, there is provided substantially pure capecitabine, wherein the amount of each of the impurities listed in Table 1 is less than about 0.1%, as determined using HPLC. The relative retention time (RRT) values are based on capecitabine=1, are typical in the above method, and can vary somewhat due to specific instruments, conditions, and operator technique. The table also shows a representative limit of detection (LOD) and limit of quantification (LOQ) for each impurity, expressed as percent. Impurity 1 and impurity 2 are analyzed together.

TABLE 1

| Impurity | Structure | RRT | LOD/LOQ |
|---|---|---|---|
| Impurity 1 | 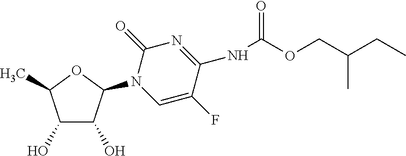 | 0.95 | 0.01/0.003 |
| Impurity 2 | 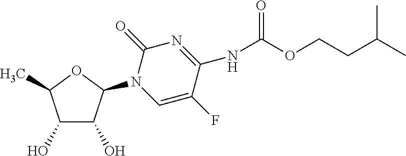 | 0.95 | |
| Related compound A (RC-A) | 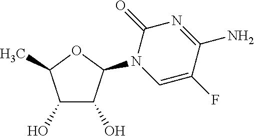 | 0.18 | 0.02/0.01 |
| Related compound B (RC-B) | 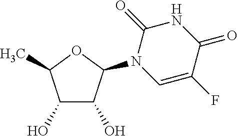 | 0.19 | 0.02/0.01 |
| Related compound C (RC-C) | 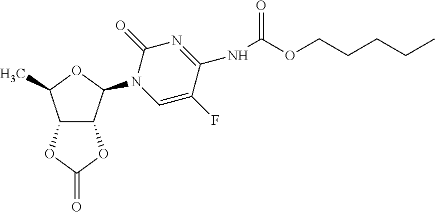 | 1.11 | 0.01/0.004 |
| Impurity 3 | 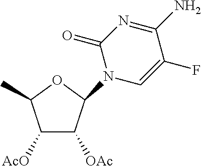 | 0.36 | 0.01/0.005 |
| Impurity 4 | 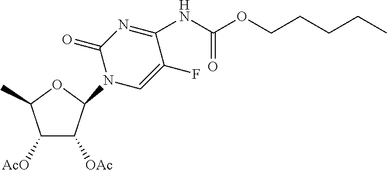 | 1.37 | 0.02/0.01 |
| Impurity 5 | 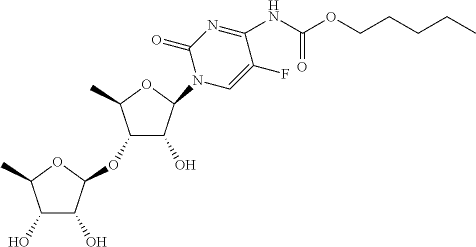 | 1.06 | 0.01/0.004 |
| Impurity 6 | 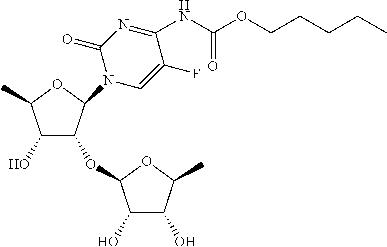 | 1.09 | 0.01/0.004 |
| Impurity 7 | 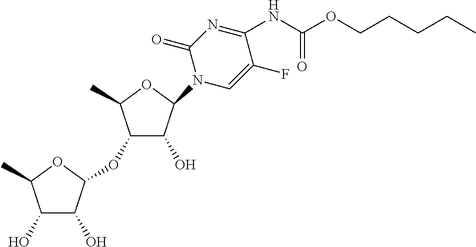 | 1.20 | 0.01/0.004 |

In embodiments, capecitabine obtained by the present application may have particle size distributions with any one or more of: D₉₀ less than about 100 μm, or less than about 60 μm; D₅₀ less than about 50 μm, or less than about 25 μm; and D₁₀ less than about 25 μm, or less than about 10 μm. D values indicate that specific fractions of particles in a sample (e.g., 90, 50, or 10 percent) have sizes less than or equal to the specified value. Particle size distributions can be measured using various techniques, including laser light scattering.

Capecitabine obtained by the present application, unless stated otherwise, can be characterized by X-ray powder diffraction (“XRPD”) patterns, differential scanning calorimetry (“DSC”) curves, and thermogravimetric analysis (TGA) curves.

All XRPD data reported herein were obtained using a Bruker AXS D8 Advance Powder X-ray Diffractometer, equipped with Bragg-Berntano Φ:Φ goniometer. Samples were ground gently and filled into a sample holder by a top loading method, and exposed to copper Kα radiation. Since some margin of error is possible in the assignment of 2-theta angles and d-spacings, a preferred method of comparing X-ray powder diffraction patterns in order to determine the presence of a particular crystalline form is to overlay the X-ray powder diffraction pattern of the unknown form over the X-ray powder diffraction pattern of a known form. For all analytical data discussed in this application, it should be kept in mind that specific values can depend on many factors, e.g., the specific instrument, sample preparation and individual operator.

DSC analysis was carried out using a DSCQ100V8.2 Build 268 instrument. The thermogram was recorded from 20 to 160° C. under a nitrogen flow of 50 mL/minute at a heating rate of 5° C./minute. About 2-4 mg of sample was placed on an aluminum pan and the sample was distributed uniformly as a thin layer.

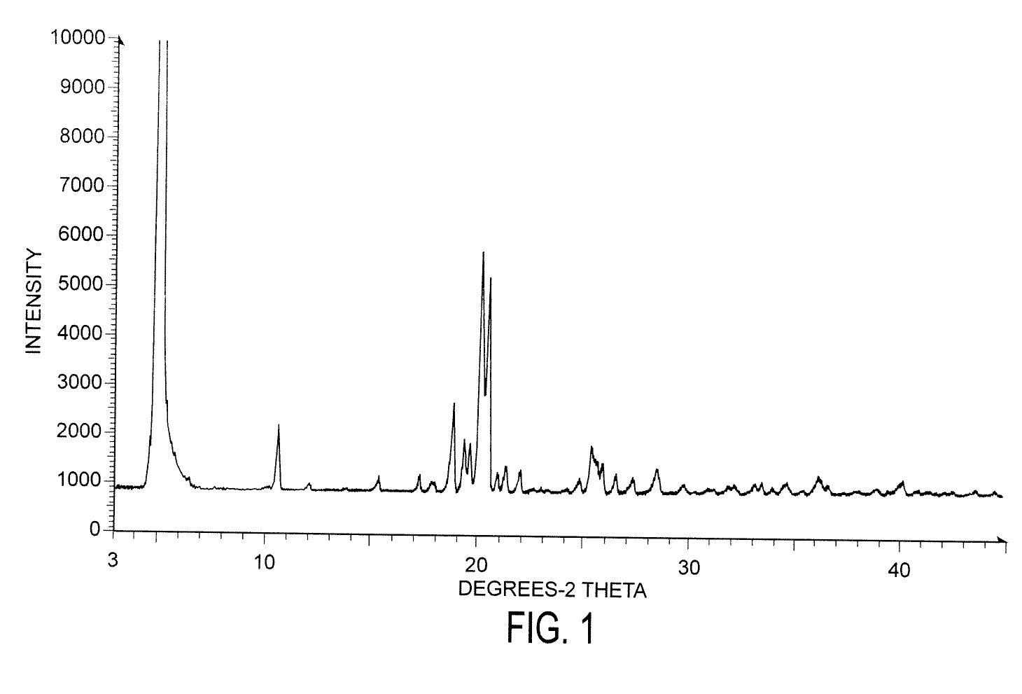

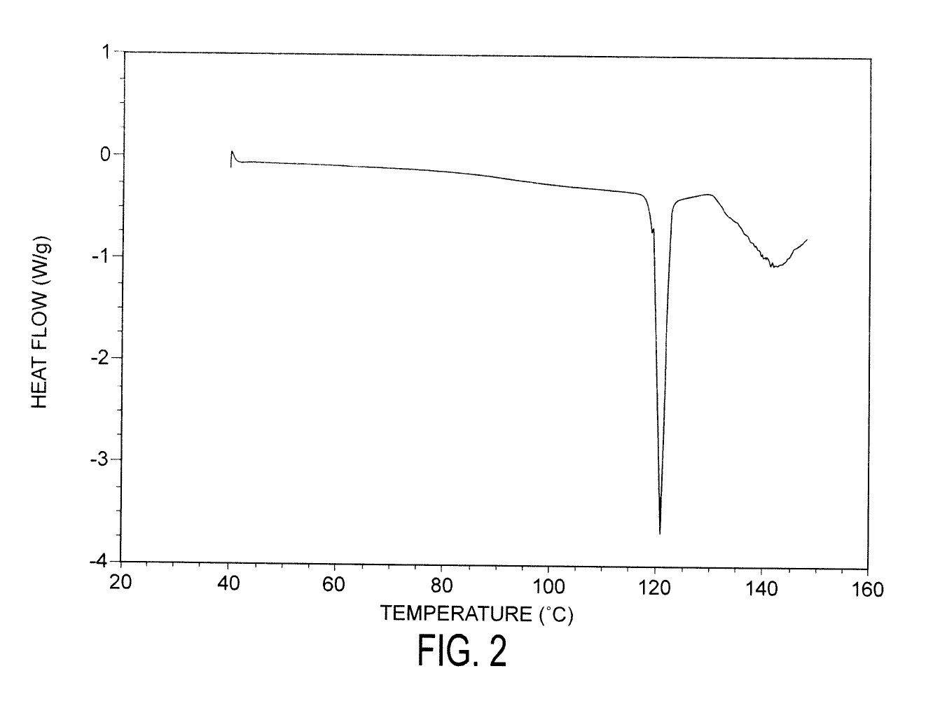

The capecitabine obtained from processes described herein and having an XRPD pattern substantially in accordance with FIG. 1 is separately contemplated. Capecitabine also can be characterized by its DSC thermogram, such as that shown in FIG. 2, having an endothermic peak at about 120° C.

The present invention includes pharmaceutical compositions comprising substantially pure capecitabine produced by the processes of the present invention, together with at least one pharmaceutically acceptable excipient.

As is known in the art, pharmaceutical compositions may be formulated as: liquid compositions for oral administration including, for example, solutions, suspensions, syrups, elixirs, and emulsions; compositions for parenteral administration, such as, suspensions, emulsions, or aqueous or non-aqueous sterile solutions; and solid oral dosage forms, such as filled hard gelatin capsules, compressed tablets, and gel caps, wherein the capecitabine is suspended, dissolved, dispersed, or emulsified in a vehicle surrounded by a soft capsule material.

Pharmaceutically acceptable excipients that are of use in the present invention include, but are not limited to: diluents such as starches, pregelatinized starches, lactose, powdered celluloses, microcrystalline celluloses, dicalcium phosphate, tricalcium phosphate, mannitol, sorbitol, sugar, and the like; binders, such as acacia, guar gum, tragacanth, gelatin, polyvinylpyrrolidones, hydroxypropyl celluloses, hydroxypropyl methyl celluloses, pregelatinized starches, and the like; disintegrants, such as starches, sodium starch glycolate, pregelatinized starches, crospovidones, croscarmellose sodium, colloidal silicon dioxide, and the like; lubricants, such as stearic acid, magnesium stearate, zinc stearate, and the like; glidants, such as colloidal silicon dioxide and the like; solubility or wetting enhancers, such as anionic, cationic, and neutral surfactants, complex forming agents, such as various grades of cyclodextrins; release rate controlling agents, such as hydroxypropyl celluloses, hydroxymethyl celluloses, hydroxypropyl methylcelluloses, ethyl celluloses, methyl celluloses, various grades of methyl methacrylates, waxes, and the like. Other pharmaceutically acceptable excipients that are of use include, but are not limited to, film formers, plasticizers, colorants, flavoring agents, sweeteners, viscosity enhancers, preservatives, antioxidants, and the like.

Certain specific aspects and embodiments of the invention will be explained in more detail with reference to the following examples, which are provided for purposes of illustration only and should not be construed as limiting the scope of the invention in any manner.

### Example 1

### Preparation of 5-deoxy-D-ribofuranose triacetate of Formula IV

D-ribose (150 g) is suspended in methanol (600 mL) and acetone (600 mL) at room temperature with stirring, and concentrated hydrochloric acid (15 mL) is added. The solution is heated to 55-60° C. and stirred for 4 hours, then is cooled to 25-30° C. and pH is adjusted to about 7.2, using 10% sodium carbonate solution (95 mL). The mixture is concentrated completely at 45° C. and then is cooled to 25-30° C. Toluene (600 mL) is added and the layers are separated. The aqueous layer is extracted with toluene (300 mL). The combined organic layer is washed with brine solution (300 mL) to yield a solution containing 97.11% pure methyl-2,3-O-isopropyl idene-D-ribofuranoside.

Triethylamine (335 mL) is added to the above solution at room temperature. A solution of 217 g of tosyl chloride dissolved in 390 mL of toluene is added over 2 hours and maintained for 10 hours. Water (975 mL) is added and stirred for 15 minutes. The layers are separated and the aqueous layer is extracted with toluene (195 mL). The combined organic layer is washed with water (975 mL), and concentrated under vacuum below 45° C. until no solvent is distilled. The gummy residue is stripped of residual solvent by adding isopropyl alcohol (195 mL) and distilling to dryness. The solid residue is dissolved in isopropyl alcohol (1170 mL) at 45° C., cooled to 0-5° C., and stirred for 2 hours. The suspension is filtered. The solid is washed with precooled isopropyl alcohol (195 mL) and dried at 40° C. for 4-5 hours, to obtain 180 g of methyl-2,3-O-isopropylidene-5-O-tosyl-D-ribofuranoside.

Methyl-2,3-O-isopropylidene-5-O-tosyl-D-ribofuranoside (150 g) produced above and sodium borohydride (23.56 g) are suspended in dimethylsulfoxide (600 mL) under a nitrogen atmosphere. The reaction mixture is stirred for 4-5 hours at 80-85° C., then is cooled to 10-15° C., quenched with 5% acetic acid solution (900 mL) below 15° C., and stirred for 15 minutes at 25-30° C. The mixture is extracted with toluene (450 mL and 300 mL) and the combined organic layer is washed with brine solution (300 mL) to obtain a solution containing methyl-2,3-O-isopropyl idene-5-deoxy-D-ribofuranoside.

Methyl-2,3-O-isopropylidene-5-deoxy-D-ribofuranoside solution (78 g), obtained above, and 0.04N sulfuric acid solution (468 mL) are combined and stirred for 15 minutes. The mixture is heated to 80-85° C., maintained for 10 hours, and then cooled to room temperature. The layers are separated and the aqueous layer is adjusted to pH 4.43 with 10% sodium carbonate solution (20 mL). The mixture is concentrated until no water distills. Toluene (157.5 ml) is added and the mixture is concentrated completely and cooled to room temperature. Toluene (157.5 ml) and trimethylamine (235.5 ml) are added and the mixture is cooled to 10-15° C. Acetic anhydride (172.5 ml) is added and the temperature is raised to 25-30° C., with stirring for 2 hours. Water (393 ml) is added and stirred for 15 minutes. The layers are separated and the aqueous layer is extracted with toluene (393 ml). The combined organic layer is washed sequentially with 10% hydrochloric acid solution (237 ml) and saturated sodium bicarbonate solution (237 ml). The organic layer is washed with water (237 ml) and is concentrated completely below 45° C. under vacuum, to obtain 60 g of 5-deoxy-D-ribofuranose triacetate of Formula IV.

### Example 2

### Preparation of N,O-(disilylated)-5-fluorocytosine and 2′,3′-di-O-acetyl-5′-deoxy-5-fluorocytidine of Formula III

5-fluorocytosine (2.32 g), HMDS (hexamethyldisilazane) (3.75 mL), and TMS-Cl (trimethylsilyl chloride) (0.4 mL) are suspended in toluene (15 mL) and heated to 110° C. The mixture is stirred for 30 minutes and cooled to 50-55° C., then is distilled until no solvent distills at 50-60° C. under high vacuum. Dichloromethane (100 mL) is charged to the residue at room temperature and the mixture is cooled to 0-5° C. 5-deoxy-D-ribofuranose triacetate of Formula IV (5 g) and SnCl₄ (2.4 mL) are added and the temperature is raised to 25-30° C. The mixture is stirred for 90 minutes. Sodium bicarbonate (8.1 g) and water (2.5 mL) are added, followed by stirring for about 2 hours. The mixture is filtered and the filtrate is washed with 5% sodium bicarbonate solution (25 mL). The obtained clear organic layer is concentrated completely under vacuum below 45° C. The residue is stripped with isopropyl alcohol (5 mL). Isopropyl alcohol (12.5 mL) is charged to the obtained solid and heated to 40-50° C. The mixture is cooled to room temperature and stirred for 15 minutes, then is further cooled to 0-5° C. and stirred for 90 minutes. The obtained suspension is filtered and the solid is washed with isopropyl alcohol (2.5 mL). The solid is dried at 47° C. under vacuum for 4-5 hours, to afford 3.7 g of the title compound.

Purity: 98.28%; impurity at 1.56 RRT: 0.73%.

2′,3′-di-O-acetyl-5′-deoxy-5-fluoro-cytidine of Formula III (5 g) obtained above is dissolved in acetonitrile (9 mL) at 50° C. with stirring for 15 minutes. The solution is cooled to room temperature and stirred for 2 hours, then is cooled to 0-5° C. and stirred for 1 hour. The obtained suspension is filtered and the solid is dried for 3 hours at 40-45° C. under vacuum, to afford 2.9 g of the title compound.

Purity: 99.56%; impurity at 1.56 RRT: 0.35%.

### Example 3

### Preparation of 2′,3′-di-O-acetyl-5′-deoxy-5-fluoro-N-[(pentyloxy)carbonyl]-cytidine of Formula II

2′,3′-di-O-acetyl-5′-deoxy-5-fluorocytidine of Formula III (15 g) is dissolved in dichloromethane (75 mL) at room temperature with stirring. Pyridine (6.1 mL) is added and the mixture is cooled to −5° C. to 10° C. N-pentylchloroformate (10.23 mL) is added slowly below 10° C. over 20 minutes and the mixture is stirred for 30 minutes at room temperature. Methanol (0.9 mL) and water (30 mL) are added and stirred for 15 minutes. The layers are separated and the obtained organic layer is washed with water (30 mL). The organic layer is concentrated completely under vacuum at 40-45° C. and then diisopropyl ether (2×30 mL) is charged and distilled completely, to obtain a product having 0.30% of the impurity at 1.14 RRT.

Acetonitrile (6 mL) and diisopropyl ether (75 mL) are charged to the product and the mixture is heated to 35° C., stirred for 1 hour at 35-40° C., then cooled to room temperature and stirred for 1 hour. The mass is filtered and the solid is washed with diisopropyl ether (15 mL). The solid is dried for 4 hours at 40° C. under vacuum, to afford 14.7 g of the title compound.

Purity: 99.8%; impurity at 1.14 RRT: 0.05%.

### Example 4

### Preparation of Capecitabine

2′,3′-di-O-acetyl-5′-deoxy-5-fluoro-N-[(pentyloxy)carbonyl]-cytidine of Formula II (5 g) is dissolved in methanol (10 mL) at room temperature and then cooled to −5 to 0° C. 1N sodium hydroxide solution (15.9 mL) is added slowly at −5 to 0° C. and stirred for 30 minutes. The pH of the mixture is adjusted to about 4.3 with concentrated hydrochloric acid (1.5 mL) at −5 to 5° C. Dichloromethane (30 mL) is added and stirred for 15 minutes at room temperature. The layers are separated and the aqueous layer is extracted with dichloromethane (20 mL) followed by washing the organic layer with water (10 mL). The combined organic layer is distilled to about 8 mL at 40-45° C. Toluene (40 mL) is added and stirred for 2 hours at 25-30° C. The suspension is filtered. The solid is washed with toluene (5 mL) and dried for 4 hours at 40-45° C. under vacuum, to give 2.7 g of title compound.

Purity: 99.78% by HPLC; impurity at 1.23 RRT: 0.1%; impurity at 0.75 RRT: 0.02%.

### Example 5

### Purification of Capecitabine

Capecitabine (44 g) (purity: 99.8%; impurity at 0.75 RRT: 0.04% and impurity at 1.09 RRT: 0.05%) is dissolved in dichloromethane (66 mL) at room temperature and stirred for 10 minutes. Toluene (440 mL) is added over 10 minutes with stirring. The suspension obtained is stirred for 2 hours and is filtered. The solid is washed with toluene (88 mL) and dried under vacuum for 4 hours below 45° C., to give 42 g of capecitabine.

Purity: 99.96%; impurity at 0.75 RRT: 0.04%.

### Example 6

### Purification of Capecitabine

Capecitabine (purity 99.8%, related compound B: 0.05%; 40 Kg) is dissolved in a mixture of dichloromethane and toluene (1:1 by volume, 240 L) at 35° C. and stirred for 15 minutes. The solution is filtered, cooled to 10-15° C. in 60 minutes, pure capecitabine seed (400 g) is added and the mixture is maintained for 2 hours. Toluene (360 L) is added over 45 minutes with stirring and maintained for 20-30 minutes. The suspension is filtered, the solid is washed with chilled toluene (80 L), and the solid is dried under vacuum for 4 hours below 40° C., to give 36.9 Kg of capecitabine.

Purity: 99.9% by HPLC.

Particle size distribution: D₁₀: 4.785 μm; D₅₀: 13.708 μm; D₉₀: 49.466 μm.

XRD pattern is as shown in FIG. 1.

DSC curve is as shown in FIG. 2.

### Example 7

### Purification of Capecitabine

Capecitabine (70 g) is dissolved in a mixture of dichloromethane and toluene (1:1 by volume, 420 mL) at 35° C. and stirred for 15 minutes. The solution is filtered, cooled to 15-20° C. in 60 minutes and maintained for 2 hours. Toluene (630 mL) is added over 45 minutes with stirring and maintained for 20-30 minutes. The suspension is filtered, the solid is washed with chilled toluene (140 mL) and the solid is dried under vacuum for 4 hours below 40° C., to give 65.5 g of capecitabine.

Purity: 99.92% by HPLC.

### Example 8

### Comparison of Impurity Profiles

A powdered XELODA tablet (150 mg capecitabine) in an amount equivalent to about 60 mg of capecitabine, is placed into a 100 mL volumetric flask, dissolved in a diluent mixture of water, methanol and acetonitrile 60:35:5 (by volume), filtered and analyzed by HPLC. For comparison, three different batches of capecitabine, obtained according to a process of the present application and purified according to Example 5 (Batches I and II), or Example 7 (Batch III), are also analyzed. Results are shown in Table 2, where the values are percentages of label capecitabine content.

TABLE 2

| | | | | | Batch | Batch |
|---|---|---|---|---|---|---|
| Impurity | RRT | XELODA* | XELODA** | Batch I | II | III |
| RC-A | 0.18 | 0.14 | 0.10 | ND | ND | ND |
| RC-C | 1.11 | 0.02 | 0.01 | ND | ND | ND |
| Impurity 1 + | 0.95 | 0.26 | 0.27 | ND | ND | 0.04 |
| Impurity 2 | | | | | | |
| Impurity 6 | 1.09 | 0.02 | 0.03 | ND | ND | ND |

*Batch No. U04000-52.
**Batch No. U8416042010.
ND: not detected.

1. A process for preparing substantially pure capecitabine comprising:
a) coupling 5-deoxy-D-ribofuranose triacetate of Formula IV, 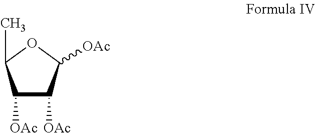
with N,O-(disilylated)-5-fluorocytosine, in the presence of a coupling catalyst and an organic solvent, to give 2′,3′-di-O-acetyl-5′-deoxy-5-fluorocytidine of Formula III, 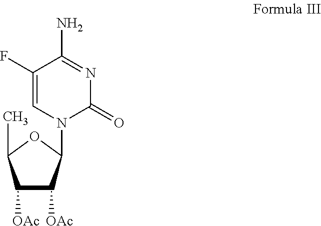
followed by purification;
b) acylating 2′,3′-di-O-acetyl-5′-deoxy-5-fluoro-cytidine with n-pentyl chloroformate in the presence of a base, to give 2′,3′-di-O-acetyl-5′-deoxy-5-fluoro-N-[(pentyloxy)carbonyl]-cytidine of Formula II, 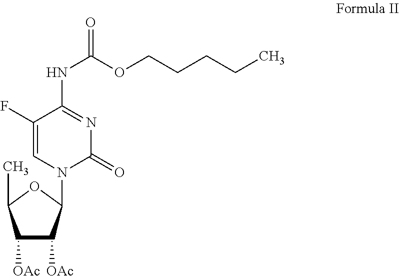
followed by slurrying the compound of Formula II in an organic solvent and isolating a purified compound of Formula II;
c) deprotecting the 2′,3′-di-O-acetyl-5′-deoxy-5-fluoro-N-[(pentyloxy)carbonyl]-cytidine of Formula II using a base in the presence of an alcohol, to give capecitabine; and
d) optionally, purifying the capecitabine.

2. The process of claim 1, wherein an organic solvent in a) comprises dichloromethane.

3. The process of claim 1, wherein a coupling catalyst comprises stannic chloride.

4. A process for purifying 2′,3′-di-O-acetyl-5′-deoxy-5-fluorocytidine of Formula III, comprising:
i) providing a solution of 2′,3′-di-O-acetyl-5′-deoxy-5-fluorocytidine in a nitrile;
ii) crystallizing a solid; and
iii) recovering purified 2′,3′-di-O-acetyl-5′-deoxy-5-fluorocytidine.

5. The process of claim 4, wherein purified 2′,3′-di-O-acetyl-5′-deoxy-5-fluorocytidine has a purity about 99.5 percent by weight, as determined using HPLC.

6. The process of claim 4, wherein a nitrile comprises acetonitrile.

7. The process of claim 4, wherein crystallizing comprises cooling to temperatures below a temperature of solution formation.

8. A purification process for 2′,3′-di-O-acetyl-5′-deoxy-5-fluoro-N-[(pentyloxy)carbonyl]-cytidine of Formula II, comprising:
i) suspending 2′,3′-di-O-acetyl-5′-deoxy-5-fluoro-N-[(pentyloxy)carbonyl]-cytidine in a liquid; and
ii) recovering a purified solid from i).

9. The process of claim 8, wherein a liquid comprises a nitrile, an ether, or a combination thereof.

10. The process of claim 8, wherein purified 2′,3′-di-O-acetyl-5′-deoxy-5-fluoro-N-[(pentyloxy)carbonyl]-cytidine has purity greater than or equal to 99.7 percent by weight, as determined using HPLC.

11. The process of claim 1, wherein an alcohol in c) comprises one or more of methanol, ethanol, isopropanol, and n-butanol.

12. The process of claim 1, wherein a base in c) comprises sodium hydroxide.

13. The process of claim 1, wherein a purification of capecitabine includes dissolving in a halogenated hydrocarbon and adding an anti-solvent.

14. The process of claim 13, wherein a halogenated hydrocarbon comprises dichloromethane.

15. The process of claim 13, wherein an anti-solvent comprises toluene.

16. A process for purifying capecitabine, comprising:
i) providing a solution of capecitabine in an organic solvent;
ii) crystallizing a solid by adding an anti-solvent; and
iii) recovering purified capecitabine.

17. The process of claim 16, wherein an organic solvent comprises a halogenated hydrocarbon.

18. The process of claim 16, wherein an anti-solvent comprises an aromatic hydrocarbon.

19. The process of claim 16, wherein purified capecitabine has a purity equal to or great than about 99.8 percent, as determined using HPLC.

20. The process of claim 16, wherein purified capecitabine has a purity at least about 99.9 percent, as determined using HPLC.

21. Capecitabine, having less than about 0.1 percent by weight of Impurity 8. [chemical structure]
